(19) United States
(12) Patent Application Publication
(10) Pub. No.: US 2026/0166057 A1
(43) Pub. Date: Jun. 18, 2026

(54) 1,3,5(10),16-ESTRATETRAEN-3-YL ACETATE FOR USE IN IMPROVING PSYCHOMOTOR OR COGNITIVE PERFORMANCE

(71) Applicant: VISTAGEN THERAPEUTICS, INC., South San Francisco, CA (US)

(72) Inventors: Louis MONTI, South San Francisco, CA (US); Reid G. Adler, South San Francisco, CA (US); Ross A. Baker, South San Francisco, CA (US)

(21) Appl. No.: 19/107,953

(22) PCT Filed: Jun. 3, 2024
(86) PCT No.: PCT/US2024/032273

§ 371 Date: Feb. 28, 2025

Related U.S. Application Data
(60) Provisional application No. 63/470,933, filed on Jun. 4, 2023.

(51) Int. Cl. A61K 31/565; A61K 9/00; A61K 31/57; A61P 25/24; A61P 25/28

(52) U.S. Cl. CPC A61K 31/565; A61K 9/0043; A61K 31/57; A61P 25/24; A61P 25/28

(57) ABSTRACT

Intranasal administration of 1,3, 5(10), 16-estratetraen-3-yl acetate is useful in improving psychomotor and/or cognitive performance in mentally fatigued persons.

### TECHNICAL FIELD

This invention relates to improving psychomotor and/or cognitive performance in mentally fatigued persons by the intranasal administration of 1,3,5(10),16-estratetraen-3-yl acetate.

### BACKGROUND ART

### Psychomotor and Cognitive Performance

According to Wetherell, “Cognitive And Psychomotor Performance Tests and Experimental Design in Multiple Chemical Sensitivity”, Environ. Health Perspect., 105 (Suppl. 2), 495-503 (1997), psychomotor function (sometimes called perceptual-motor function) refers to a person's ability to coordinate timely and appropriate responses to stimuli, while cognitive function (sometimes called cognition) refers to a person's ability to think and reason in terms of temporal and spatial relationships and in symbols such as words and numbers. In lay terms, psychomotor function (or psychomotor performance) is an ability to react. In contrast, cognitive function (or cognition or cognitive performance) is an ability to think.

As explained by Wetherell, psychomotor and cognitive performance are not discrete; they overlap to the extent that the stimuli require thought. Thus, a simple reaction time test is usually considered a test of psychomotor performance, but if the stimuli used are complex and require decisions about how to respond, the test becomes more of a test of cognitive performance. Wetherell describes a number of automated performance tests used at the UK Defence Evaluation and Research Agency Chemical and Biological Defence Human Studies Group, which he describes as all being in the public domain. He notes that these tests are used in assessing environmental stressors (primarily drugs, but also physical fatigue, sleep deprivation, and protective clothing).

Tests of psychomotor performance are primarily automated tests. The psychomotor vigilance task (PVT) is a sustained-attention, reaction-timed task that measures the speed with which a subject responds to a visual stimulus (light): the subject presses a button as soon as the light appears. The light will turn on pseudo-randomly every few seconds for 5-10 minutes. The main measurement of this task is not to assess the subject's reaction time as such, but to see how many times the button is not pressed when the light is on or pressed when the light is not on. The purpose of the PVT is to measure sustained attention/vigilance and give a numerical measure of lack of attention/vigilance by counting the number of lapses in attention of the tested subject.

Cogstate, New Haven, Connecticut, US provides a variety of computer-implemented individual tests (Cogstate refers to them as “tasks”) which can be assembled into a test battery, including a detection test (in which a card is shown face-down, then turned face-up, and the test subject is required to respond when the card is turned face-up), which is a version of the PVT. The Reaction Time Test in Example 4 is an automated psychomotor performance test.

Tests of cognitive performance include both manually scored and automated tests. Automated tests of cognitive performance include, for example, tests from Cogstate and the IntegNeuro™ system from BrainClinics Products, Nijmegen, The Netherlands. Cogstate tests include an identification test (in which a card is shown face-down, then turned face-up, and the subject is required to indicate whether the face of the card is red or black), a one-card learning test (in which cards are turned face-up one after another, with one card being shown more than once during the tests, and the subject is required to indicate whether the card being shown is new or has been previously shown), and a one-back test (where cards are turned face-up one after another, with some cards being shown twice in a row, and the subject is required to indicate whether the card being shown is new or was shown immediately before).

The IntegNeuro™ system is a touch screen computer which provides a battery of tests, including a timing test (where a circle lights up for 1 to 12 seconds, after which the subject is required to estimate the duration of the light), a choice reaction time test (where one of four circles lights up and the subject is required to touch the lit circle), a verbal interference test (where a word that is the name of a color appears in a different color on the screen and the test subject is required to indicate the color in which the word appears and not the word itself), and a “spot the real word” test (where a real word and a nonsense word are shown, and the subject is required to indicate the real word). The Time Estimation Test in Example 4 is an automated cognitive performance test.

### Mental Fatigue

Mental fatigue (in contrast to physical, or muscle, fatigue, which is the temporary physical inability of a muscle to perform optimally), is a temporary inability to maintain levels of psychomotor and/or cognitive performance that are typical for a given person or otherwise are normal or optimal for that person or for people in general. It also may result from prolonged cognitive activity (overlong activity at tasks requiring thinking or mental effort), sleep deprivation (the condition of not having enough sleep), sleep disruption or sleep apnea (which lead to sleep deprivation), or from conditions associated with these, such as shift work sleep disorder and excessive daytime sleepiness.

Mental fatigue may also result from psychological conditions such as depressive disorders, e.g., Major Depressive Disorder (MDD) and peripartum depression, and from other disorders in which mental fatigue is a typical symptom or is otherwise associated, e.g., attention deficit hyperactivity disorder and mild cognitive impairment. For example, according to Takeda et al, “Impact of depression on mental fatigue and attention in patients with multiple sclerosis”, J. Affect. Disord. Rep., 5, 100143 (2021), mental fatigue is strongly correlated with depression in patients with multiple sclerosis. However, according to Pan, et al., “Cognitive Impairment in Major Depressive Disorder”, CNS Spectrums, 24, 22-29 (2019), “Most antidepressants have not been developed and/or evaluated for their ability to ability to directly and independently ameliorate cognitive deficits”. According to Polosan et al., “Cognition—the core of major depressive disorder”, L'Encephale, 42(1, Suppl. 1), 1S3-1S11 (2016), “Cognitive deficits have been only recently recognized as a major phenotype determinant of major depressive disorder.” Pan et al. have also reported that “ . . . cognitive subdomains such as learning and memory, executive functioning, processing speed, and attention and concentration are significantly impaired during, and between, episodes in individuals with MDD.”. Moreover, depression is also highly associated with sleep disturbances (Murphy and Peterson, “Sleep Disturbances in Depression”, Sleep Med. Clinics, 10(1), 17-23 (2015)), which may contribute to cognitive deficits in major depressive disorder. Additionally, Sommerfeldt et al., “Executive Attention Impairment in Adolescents with Major Depressive Disorder,” J. Clin. Child Adolesc. Psychol., 45(1), 59-83 (2016), reported “attention impairment” in adolescents with MDD, characterized, for example, by having longer reaction times compared with controls.

Fatigue, including mental fatigue, is a common clinical feature of attention deficit hyperactivity disorder (ADHD) in adulthood according to Rogers et al., “Fatigue in an adult attention deficit hyperactivity disorder population: A trans-diagnostic approach”, Br. J. Clin. Psychol., 56(1), 33-52 (2017).

Mental fatigue may also result from (or is associated with) mild cognitive impairment (MCI). MCI is the stage between the expected decline in memory and thinking that happens with age and the more serious decline of dementia and may further be associated with depression, as noted below. MCI may include problems with memory, language or judgment, but it is not a definitive precursor to dementia, as can occur in those with other psychiatric (e.g., depression) or systemic disorders (e.g., diabetes or obesity). The Alzheimer's Association (https://www.alz.org/alzheimers-dementia/what-is-dementia/related_conditions/mild-cognitive-impairment) says of MCI that “Mild cognitive impairment (MCI) is an early stage of memory loss or other cognitive ability loss (such as language or visual/spatial perception) in individuals who maintain the ability to independently perform most activities of daily living.” They also say that “Mild cognitive impairment causes cognitive changes that are serious enough to be noticed by the person affected and by family members and friends but do not affect the individual's ability to carry out everyday activities. MCI can develop for multiple reasons, and individuals living with MCI may go on to develop dementia; others will not. For neurodegenerative diseases, MCI can be an early stage of the disease continuum including for Alzheimer's if the hallmark changes in the brain are present. The causes of MCI are not yet completely understood. Experts believe that many cases—but not all—result from brain changes occurring in the very early stages of Alzheimer's or other neurodegenerative diseases that cause dementia.

The risk factors most strongly linked to MCI when the underlying cause is neurodegenerative disease and not another cause are advancing age, family history of Alzheimer's or another dementia, and conditions that raise risk for cardiovascular disease . . . . Approximately 12% to 18% of people age 60 or older are living with MCI. An estimated 10% to 15% of individuals living with MCI develop dementia each year. About one-third of people living with MCI due to Alzheimer's disease develop dementia within five years.”

Tests for MCI include cognitive assessments such as the Mini-Cog (a composite of 3-word recall and clock drawing), General Practitioner Assessment of Cognition (GPCOG: a patient section assesses aspects of orientation, awareness, and memory; while an informant section compares patient's current and previous functioning), Montreal Cognitive Assessment (MoCA, a 1-page, 30-point test, which assesses 8 cognitive domains through 13 tasks), or Saint Louis University Mental Status Examination (SLUMS, a 30-point, 11-item scale comprising various cognitive assessments; tasks assess attention, numeric calculation, immediate and delayed recall, animal naming, digit span, clock drawing, figure recognition/size differentiation, and immediate recall of facts from a paragraph). Moreira et al., “Distinguishing mild cognitive impairment from healthy aging and Alzheimer's Disease: The contribution of the INECO Frontal Screening (IFS)”, PLoS ONE, 14(9), e0221873 (2019), report that IFS can differentiate MCI patients from cognitively healthy controls and mild to moderate Alzheimer's Disease patients. Petersen et al, “Practice guideline update summary: Mild cognitive impairment”, Neurology, 90(3), 126-135 (2018), discuss the diagnosis and treatment of MCI, but note that, while a number of drugs have been used, “No high-quality evidence exists to support pharmacologic treatments for MCI.” MCI is also known to be associated with high blood pressure and with depression and other psychiatric disorders. MCI is well-known to be distressing to persons suffering from it, and MCI may therefore cause mental fatigue resulting from that distress.

Mental fatigue may also result from combinations of any of the above situations or conditions; for example, late night “cramming” for examinations may implicate both prolonged cognitive activity and sleep deprivation; while worry and sleeplessness from MCI may implicate both sleep deprivation and the MCI itself.

The onset of mental fatigue during any cognitive activity is gradual, and depends upon a person's cognitive ability, and also upon other factors, such as existing sleep deprivation and overall health. Marcora et al., “Mental fatigue impairs physical performance in humans”, J. Appl. Physiol., 106, 857-864 (2009), have shown that mental fatigue can decrease physical performance. Mental fatigue can manifest as somnolence, lethargy, or directed attention fatigue (the inability to maintain attention or vigilance that requires a great deal of effort, as when something is monotonous or boring). Among the numerous physical consequences of mental fatigue, deficits in attention/vigilance and working memory are perhaps the most important; such deficits and consequent lapses in mundane routines can lead to unfortunate results, from forgetting ingredients while cooking to missing a sentence while taking notes.

Attentional lapses (vigilance lapses) also extend into more critical domains in which the consequences can be life-or-death; car crashes and industrial disasters can result from inattentiveness attributable to mental fatigue. The dangers of mental fatigue are particularly apparent in driving; the American Academy of Sleep Medicine (AASM) reports in its “Drowsy Driving Fact Sheet” that one in every five serious motor vehicle injuries is related to driver fatigue, with 80,000 drivers falling asleep behind the wheel every day and 250,000 accidents every year related to fatigue, though the National Highway Traffic Safety Administration suggests the figure for traffic accidents may be closer to 100,000. According to Williamson et al., “Moderate sleep deprivation produces impairments in cognitive and motor performance equivalent to legally prescribed levels of alcohol intoxication”, Occup. Environ. Med., 57(10), 649-655 (2000), mental fatigue and sleep deprivation can have some of the same hazardous effects on psychomotor and cognitive performance as being drunk.

### Countering the Effects of Mental Fatigue

Several strategies are common in attempting to counter the effects of mental fatigue.

Non-pharmacologic strategies recommended by the AASM include prophylactic sleep before sleep deprivation, naps, and combinations thereof. However, the only sure and safe way to counter mental fatigue induced by sleep deprivation/disruption is to increase nightly sleep time. According to Alhola et al., “Sleep deprivation: Impact on cognitive performance”, Neuropsychiatr. Dis. Treat., 3(5), 553-567 (2007), recovery of cognitive function is accomplished more rapidly after acute total sleep deprivation than after chronic partial sleep restriction.

The primary nonprescription drug used to counter the effects of mental fatigue is caffeine, which improves wakefulness, psychomotor function and cognitive function but does not decrease the number of mistakes during performance. Prescription drugs used to counter the effects of mental fatigue include stimulants such as amphetamine, dextroamphetamine, and the like, and eugeroics (wakefulness-promoting agents) such as modafinil and armodafinil. See, for example, Urban et al., “The Role of Eugeroics in the Treatment of Affective Disorders”, Psychiatr. Pol., 54(1), 21-33 (2020). Caffeine is often used over short periods to boost wakefulness when acute mental fatigue is experienced; however, caffeine is less effective if taken routinely, can cause jitteriness when taken in large doses, and takes some time to achieve maximal effectiveness. Amphetamines are addictive (as is caffeine) and tend to cause insomnia and emotional lability; while modafinil and armodafinil tend to cause nausea and vertigo: both also require some time to achieve maximal effectiveness and so are usually taken prophylactically before mental fatigue begins. Both amphetamines and modafinil/armodafinil are scheduled drugs in the US.

Wesensten et al., “Maintaining alertness and performance during sleep deprivation: modafinil versus caffeine”, Psychopharmacology (Berlin), 159(3), 238-247 (2002), using cognitive tests to measure wakefulness and attention level, showed that prolonged sleep deprivation was a useful method to compare the psychostimulant effect of caffeine and modafinil. However, in this study the authors did not use polygraphic recordings to evaluate the brain level of alertness and the somnolence that accompanies mental fatigue.

It would be desirable to develop an agent for improving psychomotor and/or cognitive performance in mentally fatigued persons. Such an agent will desirably be fast-acting so that it can be administered when the mental fatigue (or its effects on psychomotor and/or cognitive performance) occurs rather than needing to be taken prophylactically before expected mental fatigue. It should also have minimal side effects such as addiction, insomnia, emotional lability, and jitteriness.

### Nasal Olfactory Chemosensory Receptors, the Vomeronasal Organ and Olfactory Epithelium, and Pherines

Nasal olfactory chemosensory receptors are distributed in the olfactory epithelium, and are found in the mucosal lining of the medial and dorsal nasal septum and the dorsal nasal recess, including the vomeronasal organ (“VNO”, which is also referred to as the “Jacobson's organ”). In particular, such receptors in the VNO have been associated with pheromone reception in many non-human species (generally see Monti-Bloch et al., “Effect of putative pheromones on the electrical activity of the human vomeronasal organ and olfactory epithelium”, J. Steroid Biochem. Mol. Biol., 39(4):573-582 (1991); and Monti-Bloch et al., “The Human Vomeronasal System: A Review”, Ann. NY Acad. Sci., 855:373-389 (1998). The axons of the neuroepithelia of the nasal chemosensory receptors have direct input to the hypothalamus and limbic amygdala of the brain, while the distal processes (cilia and microvilli) are the sites where chemosensory receptors are located (see Stensaas et al., “Ultrastructure of the human vomeronasal organ”, J. Steroid Biochem. Mol. Biol., 39(4):553-560 (1991), and Monti-Bloch, “Patch recorded isolated adult human vomeronasal cells are electrically excitable and respond to skin steroidal substances: androsta-4,16-dien-3-one and estra-1,3,5(10),16-tetraen-3-ol”, Abstract #200 for Seventeenth Annual Meeting of the Association for Chemoreception Sciences (AChemS XVII), Chem. Senses, 20(6): 745-746 (1995).

Human derived neurosteroids delivered to the nasal septal area bind to cilia and microvilli on local chemosensory receptors and trigger nerve signals that reach the brain inducing physiological and behavioral changes (Monti et al., “Effect of Putative Pheromones On the Electrical Activity of the Human Vomeronasal Organ and Olfactory Epithelium”, J. Steroid Biochem. Molec. Biol., 39(4B), 573-582 (1991); and Grosser et al., “Behavioral and electrophysiological effects of androstadienone, a human pheromone”, Psychoneuroendocrinology, 2000, 25:289-299). Pherines, which are chemically modified naturally occurring human neurosteroids (substances that bind to nasal olfactory chemosensory receptors) can induce robust physiological, pharmacological and behavioral changes when delivered airborne to these receptors via the nasal passages. This information is supported by several studies in human volunteers using functional magnetic resonance imaging and positron emission tomography, showing that pherines selectively activate the brain areas (hypothalamus, limbic system, cingulate gyrus, anterior thalamus and prefrontal cortex) where their physiological, pharmacological and behavioral effects are integrated.

Studies with several pherines have shown that because the compounds act directly on nasal olfactory chemosensory receptors that are directly connected to the brain, administration of the compounds causes an effect on physiological markers (e.g., autonomic nervous system responses and EEG) within seconds to less than a minute, and an effect on endocrine and neurotransmitter metabolite markers within about 10-15 minutes.

### 1,3,5(10),16-Estratetraen-3-yl acetate

1,3,5(10),16-Estratetraen-3-yl acetate (“estratetraenyl acetate”, “ETA”) and its synthesis are described in, for example, Berliner et al., U.S. Pat. No. 5,783,571, “Method of altering hypothalamic function by nasal administration of estrene steroids”. This patent describes the use of a number of estranes as compounds capable of altering hypothalamic or autonomic function by administration to the olfactory epithelium of human subjects. ETA is disclosed in the patent, where it is mentioned as a preferred compound at column 8, around line 20. It is the acetate ester of 1,3,5(10),16-estratetraen-3-ol, which is mentioned as a preferred compound in the same paragraph and is compound E2/N1 in the chart of estranes (bottom of column 7, described as “known”). The patent generically claims pharmaceutical compositions containing estranes, including ETA, in dosage form adapted for nasal administration, and discloses methods of altering hypothalamic or autonomic function with them by vomeronasal administration.

ETA, administered as a 60 pg dose to the vomeronasal organ, produced a change in mass receptor potential (electrovomerogram) when compared to control (propylene glycol) that was of higher amplitude in men (FIG. 3A) than in women (FIG. 3B). ETA was also shown to produce an increase in integrated electrovomerogram (FIG. 4A), a decrease in skin resistance (FIG. 4B), and an increase in skin temperature (FIG. 4C), in men when compared to control. ETA was also shown to produce an increase in electrovomerogram (FIGS. 6A and 6B), an increase in α-cortical activity (FIGS. 6E and 6F), and an increase in skin temperature (FIGS. 6G and 6H), in both men and women, with the effect being more pronounced in men; while it produced a decrease in electrodermal activity in men (FIG. 6C) but not in women (FIG. 6D). However, this patent does not describe improvements in psychomotor or cognitive function or provide any related data.

Some later patents, such as Jennings-White et al., U.S. Pat. No. 6,057,439, “Steroids as neurochemical stimulators of the VNO to alleviate symptoms of PMS and anxiety”, Jennings-White et al., U.S. Pat. No. 6,066,627, “Steroids as neurochemical initiators of change in human blood levels of LH”, Jennings-White et al., U.S. Pat. No. 6,117,860, “Steroids as neurochemical stimulators of the VNO to treat paroxistic tachycardia”, and Berliner et al., U.S. Pat. No. 6,331,534, “Steroids as neurochemical stimulators of the VNO to alleviate pain”, have slightly expanded disclosures about ETA, while also including many other steroids of different classes.

Berliner et al., U.S. Pat. No. 6,544,971, “Method of increasing alertness by administration of a vomeropherin, and vomeropherin-emitting alarm devices”, discloses a method of increasing alertness in an individual by vomeronasal administration of an estrane of the formula:

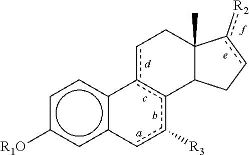

where R₁ is hydrogen, C₁₋₄ alkanoyl, or —SO₃H or a salt thereof; R₂ is hydrogen or methylene; R₃ is absent, hydrogen, or C₁₋₄ alkyl; one or two non-adjacent members of “a”, “b”, “c”, and “d” are optional double bonds; and when R₂ is hydrogen, “e” is either a double bond or a 16α,17α-epoxide; and when R₂ is methylene, “e” is absent and “f” is a double bond. The patent also discloses alarm devices for detecting the presence of an alarm condition and a dispenser for vomeronasally administering the estrane. Compounds said to be preferred are ETA and 17-methylene-1,3,5(10),6,8-estrapentaen-3-ol.

U.S. Pat. No. 6,544,971 defines “alertness” as including wakefulness and responsiveness to external stimuli. It also defines “increasing alertness in an individual” as including either or both of awakening that individual and increasing that individual's responsiveness to an external stimulus, an effect that may take place with lightening of a state of sleep but without full awakening. Increasing alertness is said to mean “increasing responsiveness of that individual to external stimuli, such as a bell ringing, phone ringing, fire, smoke and the like.” The patent also defines “alarm conditions” with respect to an individual as one in which the safety or health of that individual or others may be adversely affected by the lack of a response by that individual, or one in which there is a request for an individual to respond.

### SUMMARY OF THE INVENTION

In a first aspect, this invention is a method of improving psychomotor and/or cognitive performance in mentally fatigued persons by nasal administration of 1,3,5(10),16-estratetraen-3-yl acetate.

The mental fatigue may arise from any cause, but causes of note include prolonged cognitive activity, sleep deprivation, sleep disruption, and conditions associated with these, such as shift work sleep disorder and excessive daytime sleepiness. It may also arise from, or be associated with, psychological conditions such as depressive disorders (including Major Depressive Disorder and peripartum depression) and other disorders such as attention deficit hyperactivity disorder and mild cognitive impairment.

In other aspects, this invention thus includes:
1,3,5(10),16-estratetraen-3-yl acetate for improving psychomotor and/or cognitive performance in mentally fatigued persons by nasal administration;
pharmaceutical formulations and devices containing 1,3,5(10),16-estratetraen-3-yl acetate for improving psychomotor and/or cognitive performance in mentally fatigued persons by nasal administration; and
the use of 1,3,5(10),16-estratetraen-3-yl acetate in the manufacture of medicaments for improving psychomotor and/or cognitive performance in mentally fatigued persons by nasal administration.

1,3,5(10),16-estratetraen-3-yl acetate has particular utility in improving psychomotor and/or cognitive performance in such persons; and is expected to have the following advantages over conventional stimulants:
(1) rapid onset of effect, because of the direct local delivery of the compound to nasal olfactory chemosensory receptors and consequent action;
(2) lack of local nasal and systemic adverse effects or toxicity, because of the very low (nanogram to low microgram) doses used and the local route of administration, meaning no to low systemic uptake; and
(3) lack of the side effects seen with other commonly used agents, such as caffeine, the amphetamines, and modafinil/armodafinil, for the same reason as (2) above.

Preferred embodiments of this invention are characterized by the specification and by the features of the claims of this application as filed, and of corresponding pharmaceutical compositions, devices, methods, and uses of the compound.

### DETAILED DESCRIPTION

### Definitions

“Person” or “persons” refers to humans; “man” or “men” refers to male humans; “woman” or “women” refers to female humans; no limitation by age is intended.

“Nasal administration” or “intranasal administration” is administration to human nasal olfactory chemosensory receptors. In a clinical setting, this may be accomplished to a certain extent by the use of a probe specifically designed to administer the ETA essentially solely to the VNO (such a probe, also designed to measure the effect on vomeronasal tissue, is described in Monti-Bloch, U.S. Pat. No. 5,303,703, “Combined neuroepithelial sample delivery electrode device and methods of using same”). Preferably, however, nasal administration comprises administration to the nasal cavity via conventional nasal spray devices in a manner that directs the ETA generally towards the primary and secondary sites of olfactory chemosensory receptors in the olfactory epithelium of the nasal cavity, including the dorsal nasal recess and the VNO. See, for example, U.S. Provisional Patent Application Ser. No. 63/631,389, filed Apr. 8, 2024, and entitled “Intranasal Drug Delivery System.”

An “effective amount” means the amount of ETA that, when administered to the nasal olfactory chemosensory receptors of a mentally fatigued person is sufficient to improve psychomotor and/or cognitive performance in that person, but which amount is insufficient to have a systemic effect by absorption into the circulation.

“Improving” psychomotor and/or cognitive performance in a mentally fatigued person includes at least one of:
improving psychomotor performance, i.e., improving the ability of the person to coordinate timely and appropriate responses to stimuli, by causing psychomotor performance to be increased or enhanced compared to the person's psychomotor performance without the nasal administration of the ETA, and
improving cognitive performance, i.e., improving the ability of the person to think and reason in terms of temporal and spatial relationships and in symbols such as words and numbers, by causing cognitive performance to be increased or enhanced compared to the person's performance without the nasal administration of the ETA.

Because psychomotor and/or cognitive performance are decreased in mentally fatigued persons, i.e., mentally fatigued persons suffer from deficits in psychomotor and/or cognitive performance, “improving” psychomotor and/or cognitive performance includes reducing these deficits.

“Psychomotor performance” and “cognitive performance” and tests used for measuring them are described in the section “Psychomotor and cognitive performance” of the Background art.

“Mental fatigue” and its effects on psychomotor and/or cognitive performance are described in the section “Mental fatigue” of the Background art.

A “pharmaceutically acceptable excipient” means an excipient that is useful in preparing a pharmaceutical formulation that is generally safe, non-toxic, and desirable. These excipients may be solid, liquid, semisolid, or, in the case of an aerosol composition, gaseous.

### 1,3,5(10),16-Estratetraen-3-yl acetate and its Preparation

1,3,5(10),16-Estratetraen-3-yl acetate is the compound of the formula:

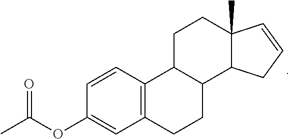 [SMILES: CC(=O)Oc1ccc2c(c1)CCC1C2CC[C@]2(C)C=CCC12]

It is easily prepared by the esterification of 1,3,5(10),16-estratetraen-3-ol, by methods such as those described in paragraph [0037] below.

The preparation of ETA is described in Examples 1 and 2 of U.S. Pat. No. 5,783,571, starting from the readily commercially available steroid estrone (3-hydroxy-1,3,5(10)-estratrien-17-one, available from a number of suppliers, such as Sigma-Aldrich Company LLC), as illustrated in the following reaction scheme:

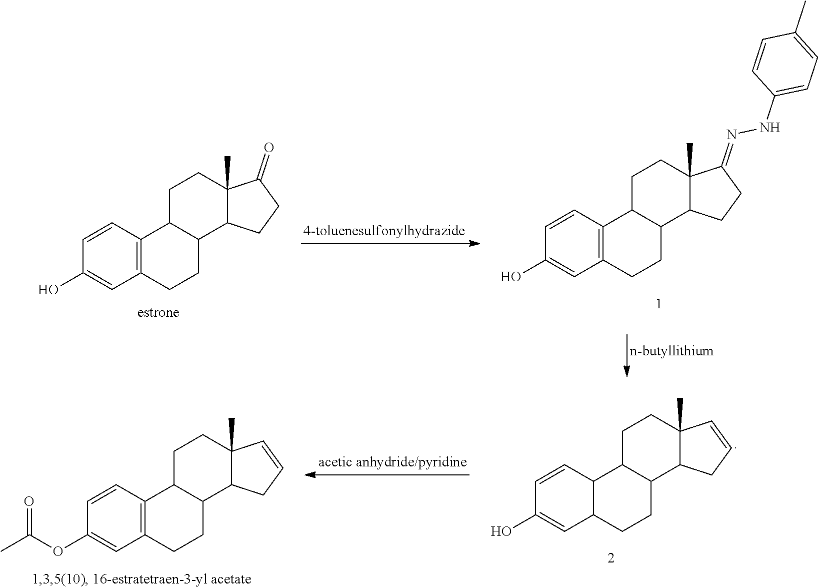 [SMILES: C[C@]12CCC3c4ccc(O)cc4CCC3C1CCC2=O ; Cc1ccc(N/N=C2\CCC3C4CCc5cc(O)ccc5C4CC[C@]23C)cc1 ; CC(=O)Oc1ccc2c(c1)CCC1C2CC[C@]2(C)C=CCC12 ; C[C@@]12C=CCC1C1CCC3C=C(O)C=CC3C1CC2]

From the description of the synthesis in Examples 1 and 2 of U.S. Pat. No. 5,783,571: In the first step, estrone (270 g, 1.00 mol) and 4-toluenesulfonylhydrazide (232.8 g, 1.25 mol) in dry methanol (2.5 L) were heated under reflux for 20 hours. The mixture was transferred to a conical flask and allowed to cool. The crystalline estrone 4-toluenesulfonylhydrazone 1 that formed was filtered off under suction and washed with methanol (300 mL). Further crops were obtained by sequentially evaporating the filtrate to 2 L, 800 mL, and 400 mL, and allowing the estrone 4-toluenesulfonylhydrazone to crystallize each time. The total yield was 433.5 g (99%).

In the second step, estrone 4-toluenesulfonylhydrazone (219.0 g, 500 mmol) in dry tetrahydrofuran (8.0 L) was cooled in a sodium chloride/ice bath. The mixture was mechanically stirred while n-butyllithium (800 mL of a 2.5M solution in hexane, 2.00 mol) was added by a double-ended needle. The mixture was stirred at room temperature for 3 days. Ice (250 g) was added, followed by saturated aqueous ammonium chloride solution (500 mL). The phases were mixed by stirring and then allowed to settle. The aqueous phase was removed by aspiration with a PTFE tube and extracted with ether (500 mL). The two organic phases were sequentially washed with the same batch of saturated aqueous sodium bicarbonate solution (500 mL) followed by saturated aqueous sodium chloride solution (500 mL). The organic layers were dried (MgSO₄) and evaporated under vacuum to give crude 1,3,5(10),16-estratetraen-3-ol 2. This was subjected to flash filtration on silica gel 60 (500 g 230-400 mesh), eluting with ethyl acetate/hexane (1:3, 2.5 L). The filtrate was evaporated under vacuum to give 1,3,5(10),16-estratetraen-3-ol as a crystalline material. This was recrystallized from methanol/water (4:1, 375 mL), washing with methanol/water (4:1, 100 mL). Further recrystallization from ethyl acetate/hexane (1:7) gave pure 1,3,5(10),16-estratetraen-3-ol (88.9 g, 70%).

In the third step, to 1,3,5(10),16-estratetraen-3-ol (254 mg, 1.00 mmol) in ether (10 mL) is added acetic anhydride (0.25 mL) followed by pyridine (0.25 mL), and the mixture is stirred at room temperature for 16 hours. The mixture is poured into ice/water and extracted with ether (2×20 mL). The organic extracts are washed with water, saturated aqueous copper sulfate solution, water, and saturated aqueous sodium chloride solution, dried (MgSO₄) and evaporated under vacuum to give crude 1,3,5(10),16-estratetraen-3-yl acetate. This is purified by flash chromatography on silica gel 60 (17.5 g, 230-400 mesh) eluting with 10%-12% ethyl acetate/hexane to give pure 1,3,5(10),16 estratetraen-3-yl acetate (192 mg, 65%).

A person of ordinary skill in the art will have no difficulty, considering that skill and this disclosure (including the patent mentioned above), in preparing ETA.

### Formulation and Administration

The ETA may be administered nasally by any suitable route designed to bring the ETA into contact with nasal olfactory chemosensory receptors. Routes of administration include, but are not limited to, topical nasal applications (e.g., of a dermal or preferably an intranasal cream or gel), nasal spray, nasal powder spray, nasal aerosol, and the like. Pharmaceutical formulations generally will be of the kind designed to administer the drug across mucosal membranes or transdermal formulations. Suitable formulations for each of these methods of administration may be found, for example, in Remington: The Science and Practice of Pharmacy, 20 ed., A. Gennaro, ed., Lippincott Williams & Wilkins, Philadelphia, Pennsylvania, U.S.A., 2003. Typical preferred formulations will be aqueous solutions for nasal spray, and will contain ETA and water, and typically will also contain one or more other pharmaceutically acceptable excipients to increase the solubility of the ETA, such as alcohols and glycols (for example, ethanol and propylene glycol). Suitable delivery devices for these formulations are the metered-dose nasal spray pumps in common use for intranasal delivery of steroids for allergies and asthma. Such pumps are made by a number of manufacturers. Liquid volumes should be such that the formulation is efficiently delivered without exceeding the nasal retention volume with an excess either flowing back into the nasal sinuses or dripping from the nose, and a volume of 50 μL has been found convenient, though greater or lesser volumes will also be satisfactory. An exemplary formulation includes the one discussed in paragraph [0057] below; and a person of ordinary skill in the art will have no difficulty, considering that skill and this disclosure, in preparing suitable formulations and delivery systems of ETA for nasal administration.

An effective amount of ETA administered intranasally, when administered in a nasal spray formulation of the type above, is about 400 to 6000 nanograms per administration, such as 1000 to 4000 nanograms per administration, for example about 1600-3200 nanograms per administration (or one-half that per nostril, assuming that the compound is administered to both nostrils), with the dose for men typically being in the lower part of the ranges given, such as from 1000 to 2500 nanograms per administration, for example about 1600 nanograms per administration, and the dose for women typically being in the higher part of the ranges given, such as from 2500 to 4000 nanograms per administration, for example about 3200 nanograms per administration. It is expected that not more than a few percent of this intranasal spray dose will actually reach the nasal olfactory chemosensory receptors, so effective amounts when administered essentially solely to the nasal olfactory chemosensory receptors may well be lower. Doses for other methods of intranasal administration may differ, depending on the method; and a person of ordinary skill in the art will have no difficulty, considering that skill and this disclosure, in preparing determining a suitable dose range for a given method of administration/formulation/delivery system. These doses, both nasal/intranasal and direct to the nasal olfactory chemosensory receptors, are well below any level that would cause a systemic effect other than those mediated through the nasal olfactory chemosensory receptors.

Initial physiological response to the nasal administration of ETA takes place very rapidly, typically within a minute of administration; and an improvement in psychomotor and cognitive performance is typically seen within 15 minutes of administration, such as within 5 minutes of administration. Because of the rapid onset of effect of nasally administered ETA, and its safety, it is expected that ETA may be administered as needed once mental fatigue is sensed, for example immediately a mentally fatigued person senses sleepiness or a symptom of sleepiness (such as excessive yawning, closing the eyes, nodding of the head, etc.) or a decline in psychomotor or cognitive performance, to improve psychomotor and cognitive performance under those circumstances. It is also expected that ETA may be administered prophylactically to improve, or at least prevent the normal decline in, psychomotor and/or cognitive performance in persons who are, or are likely to become, mentally fatigued. Thus, for example, a firefighter, whose work schedule may require him or her to be available continuously for 72 hours even though he/she is allowed to sleep at night when not actually needed, might administer ETA to improve psychomotor and/or cognitive performance when awakened at night and called to fight a fire; or a pilot of an overnight flight, who flies the aircraft during an afternoon takeoff, rests during the overnight cruise portion of the flight, but then flies the aircraft again as it approaches and lands the next morning, might administer ETA to improve psychomotor and/or cognitive performance on rising from rest to perform the approach and landing.

It is also expected that the ETA may be administered in a scheduled basis throughout a period of awakening (such as during a shift of work) or throughout an overnight or similar period of activity (such as for aircrew on an overnight flight), such as from 2 to 8 times during that period of awakening or activity, for example from 3 to 5 times, such as 4 times, to minimize the decline in psychomotor and/or cognitive performance from fatigue in a chronic fashion, such as over a period of weeks or months. This scheduled administration may be on a uniform schedule, for example, at 10 p.m., midnight, 2 a.m., and 4 a.m. (for 4 times/day administration for a night shift worker, such as a nurse), or on a non-uniform schedule where the frequency of administration is based on the circadian rhythm of mental fatigue, either in the population at large or in the person being treated. Thus, for example, administration might be at times chosen to maximize the administration at the time when the occurrence of mental fatigue (or decline in psychomotor and/or cognitive performance) is greatest or expected to be greatest. Of course, even if scheduled administration is being used, it is possible to administer the ETA on an as-needed basis if mental fatigue (or decline in psychomotor and/or cognitive performance) is still experienced.

Similarly, for persons requiring an improvement in the deficits in psychomotor and/or cognitive function associated with depressive disorders (such as Major Depressive Disorder and post-partum depression), and other disorders such as attention deficit-hyperactivity disorder, “long COVID”, shift work sleep disorder, excessive daytime sleepiness, or mild cognitive impairment, in persons suffering from these disorders, administration may be as necessary (when a decline in psychomotor and/or cognitive function is sensed), prophylactically (to prevent or ameliorate the effects of such a decline), or on a scheduled basis.

### EXAMPLES

### Example 1: Electrophysiological Studies with ETA

ETA induced inward currents in the membrane of isolated human nasal olfactory chemosensory neurons and electrotonic depolarization of the nasal septal olfactory chemosensory epithelium: the first event in chemotransduction in peripheral receptors. The amplitude of this response increased depending on the concentration of the compound. In vitro, there was no agonist or antagonist activity on rat estrogen, androgen, progestin, and glucocorticoid receptors.

### Example 2: Preclinical Studies with ETA

Genotoxicity tests revealed no evidence of mutagenic or clastogenic potential of ETA when examined in the Ames reverse mutation assay and the in vivo bone marrow micronucleus test.

Administration of ETA (1 mg/day intravenously) to rats of both sexes caused no mortality or adverse symptoms, or changes in behavior, during a 7-day period, and no macroscopic or microscopic changes were found in all organs studied. Administration of ETA (600 μg/Kg intravenous, 900 μg/Kg intranasal) to male rats caused no mortality or adverse symptoms, or changes in behavior, during a 7-day period, and showed rapid absorption, metabolism, and excretion.

### Example 3—Pilot Human Clinical Studies with ETA

In pilot trials with healthy volunteers of both sexes, the nasal administration of ETA was well tolerated and induced robust responses. Intranasal administration of the compound induced significant change in heart rate and respiratory rate, but did not change the duration of the QTc intervals of the electrocardiogram. ETA significantly increased the frequency of electrodermal activity events (measured as skin conductance, indicating increased sympathomimetic activity) but did not have significant effect on body temperature and alpha and beta frequency bands of the electroencephalogram.

### Example 4—Efficacy Study of ETA in Mentally Fatigued Men

The study was a computerized Reaction Time and Time Estimation Test (RTTET), developed by the testing laboratory. The psychomotor performance test (Reaction Time Test) consisted of the subject depressing a lever every time he or she perceived a visual stimulus (flash), allowing measurement of the reaction time to the visual stimulus. The flashes were delivered at a fixed interval of 10 seconds (isochronous stimuli), or at random intervals (stochastic stimuli). The cognitive performance test (Time Estimation Test) consisted of the subject observing three trains of flashes delivered at a fixed interval of 10 seconds, and at the end of the third train, they were to depress the lever at the same interval (i.e., estimating the 10-second inter-flash interval).

During the test, subjects need to maintain an adequate level of attention to avoid mistakes (missing a flash, delayed response) during the Reaction Time Test; and need to be attentive to estimate the duration of the time interval between stimuli in the Time Estimation Test, since they need to use this information in their response. One sequence of the RTTET lasted 20 minutes, and each test consisted of four sequences with a 10-20 minute interval between sequences. The tests were administered at 6 p.m. (control), 9 p.m., midnight, and 3 a.m., so that each session consisted of four tests. Subjects were not allowed to sleep between the tests.

The study design was a double-blind, placebo-controlled, three-way crossover study, using ten healthy male volunteers. Inclusion criteria for the study were a history of good health, a regular nocturnal sleep pattern with regular daily onset and average duration of 8 hours, and freedom from any psychiatric medications for at least two months; while exclusion criteria included hypersensitivity to or tolerance of caffeine, and any sleep, attention, or memory disorder. The subjects were tested at 8-day intervals.

The test compound, ETA, was administered intranasally: 1600 ng ETA (one administration per nostril by a Valois intranasal spray pump of 50 μL of an aqueous solution containing 16 μg/mL ETA, 2% propylene glycol, and 2% ethanol); while the comparator compound, caffeine, was administered orally: 400 mg tablet. An intranasal placebo (same pump and solution omitting the ETA) and oral placebo (lactose tablet) were also used.

Each test night began at 5 p.m. with the subject arriving at the laboratory and being fitted with electrodes for polygraph recording of sleep (electroencephalogram, electrooculogram, electromyogram) according to the criteria of A manual of standardized terminology, techniques and scoring system for sleep stages of human subjects, Rechtshaffen and Kales, eds., NIH Publication No. 204, US Government Printing Office, 1968. All subjects received an oral dose and an intranasal dose, whether of placebo or active ingredient—ETA or caffeine, as applicable, 1 hour before the beginning of each test, and received an intranasal dose 10 minutes before and 40 minutes after the beginning of each test. In the control session, all doses were placebo; in the ETA session, all oral doses were placebo, the intranasal doses 10 minutes before and 40 minutes after the beginning of the 9 p.m., midnight, and 3 a.m. tests contained ETA and the remainder were placebo; and in the caffeine session, all intranasal doses were placebo, the oral dose 1 hour before the beginning of the 9 p.m. session contained caffeine and the remainder were placebo. The dose timing was chosen to ensure maximal efficacy of the ETA and caffeine.

There were no serious adverse events; and the most frequent mild adverse events were physical tiredness on completion of the study session in the majority of subjects, and sneezing after nasal administration in one subject. ETA nasal spray was well tolerated by all subjects.

During the tests, the subjects showed polygraphic signs of somnolence or first or second stage sleep about half-way through the night. The effect of caffeine was to significantly decrease both the reaction time and the number of errors on both the isochronous and stochastic stimuli Reaction Time tests (p<0.005 and p<0.001 relative to placebo) at all three non-control tests, with the effect being more pronounced with stochastic stimuli; and, while it improved the Time Estimation test, the effect was only statistically significant (p<0.001) on the midnight test, 4 hours after caffeine administration.

The effect of ETA was to significantly decrease both the reaction time and the number of errors on both the isochronous and stochastic stimuli Reaction Time tests (p<0.001 and p<0.0001 relative to placebo) at all three non-control tests, with the effect being more pronounced with stochastic stimuli; and it significantly improved the Time Estimation test (p<0.01) at both midnight and 3 a.m. When compared to caffeine and placebo, ETA caused less variability in both the isochronous and stochastic Reaction Time tests; and, while the effect of ETA was similar to that of caffeine at the 9 p.m. test (when the subjects were relatively non-fatigued), the effect of ETA was significantly better than the effect of caffeine (p<0.001 and p<0.0001 relative to caffeine for the isochronous and stochastic tests, respectively) at the midnight and 3 a.m. tests (when the subjects were fatigued). Also, ETA significantly improved the Time Estimation test (p<0.01 relative to caffeine) at the midnight and 3 a.m. tests.

These data demonstrate the safety and efficacy of the nasal administration of 1,3,5(10),16-estratetraen-3-yl acetate in improving psychomotor and/or cognitive performance in mentally fatigued persons.

### Example 5—Improvement of Psychomotor and/or Cognitive Performance by ETA in Mental Fatigue Associated with Shift Work Sleep Disorder

According to Jang, “Work-Fitness Evaluation for Shift Work Disorder,” Int. J. Environ. Res. Public Health, 18(3), 1294 (2021), shift work sleep disorder (shift work disorder; classified in the DSM-5 as Circadian Rhythm Sleep Disorder, 307.45) is characterized by insomnia and excessive sleepiness related to shift work, and is one of the most common health problems in shift workers. As described by Valentino et al., “Modafinil in the treatment of excessive daytime sleepiness,” Cleve. Clin. J. Med., 74(8), 561-571 (2007), modafinil is approved for treating excessive daytime sleepiness associated with shift work sleep disorder. Based in part on the discovery that ETA directly improves psychomotor and cognitive performance in mentally fatigued subjects with an efficacy similar to that of caffeine and modafinil, ETA will similarly improve the psychomotor and cognitive performance of persons with mental fatigue associated with shift work sleep disorder.

In subjects experiencing decreased psychomotor and/or cognitive performance in mental fatigue associated with shift work sleep disorder, a therapeutically effective amount of ETA is intranasally administered as described in Example 4. Psychomotor and cognitive performance improve in this group of subjects.

### Example 6—Improvement of Psychomotor and/or Cognitive Performance by ETA in Mental Fatigue Associated with Excessive Daytime Sleepiness

As described by Valentino et al., modafinil also is approved for treating excessive daytime sleepiness associated with narcolepsy and as an adjunctive treatment in patients with obstructive sleep apnea syndrome who have residual daytime sleepiness despite optimal treatment with continuous positive airway pressure. Based in part on the discovery that ETA directly improves psychomotor and cognitive performance in mentally fatigued subjects with an efficacy similar to that of caffeine and modafinil, ETA will similarly improve the psychomotor and cognitive performance of persons with mental fatigue associated with excessive daytime sleepiness.

In subjects experiencing decreased psychomotor and/or cognitive performance in mental fatigue associated with excessive daytime sleepiness, a therapeutically effective amount of ETA is intranasally administered as described in Example 4. Psychomotor and cognitive performance improve in this group of subjects.

### Example 7—Improvement of Psychomotor and/or Cognitive Performance by ETA in Mental Fatigue Associated with Depressive Disorders Such as Major Depressive Disorder and Peripartum Depression

Reaction time is one of the performance aspects described in Example 4, above, as being improved by both caffeine and ETA., franpour, et al. “Inverse Association Between Caffeine Intake and Depressive Symptoms in US Adults: Data from National Health and Nutrition Examination Survey (NHANES) 2005-2006”, Psych. Res., 271, 732-739 (2019), have shown that caffeine reduces depression symptoms measured by the PHQ-9 test, a 9-item patient health questionnaire for the assessment of depressive symptoms developed by Pfizer, Inc. The test asks, for each of the 9 items, “Over the last two weeks, how often have you been bothered by any of the following problems?”, with answers scored from 0 (“Not at all”) to 3 (“Nearly every day”); giving a total score between 0 and 27. One of the questions in this test asks about “Trouble concentrating on things, such as reading the newspaper or watching television?”. Similarly, Paech et al., “Caffeine administration at night during extended wakefulness effectively mitigates performance impairment but not subjective assessments of fatigue and sleepiness”, Pharmacol. Biochem. Behavior, 145, 27-32 (2016), showed that caffeine improved both reaction time and cognitive performance.

The medical literature is generally consistent that caffeine is negatively associated with depression; in other words, caffeine reduces depression. An article by Bao et al., “Caffeine Is Negatively Associated with Depression in Patients Aged 20 and Older,” Front. Psych., 13:1037579 (2022), concludes that: “These results suggest that people can consume some caffeine to reduce depression.” Bao at al. also note that “Several epidemiological studies have found that caffeine use has a protective effect against cognitive impairment/decline . . . ”, and state that “further study is needed to examine the precise causal relationship between these factors” because of a “complex association between caffeine intake and depression risk.” However, the article explains that, because of a similar structure, caffeine is able to compete with adenosine to bind to adenosine A1 receptors (ADORA1) and adenosine A2A receptors (ADORA2A) to influence the neural network in the brain. Lopez-Cruz, et al., “Caffeine and Selective Adenosine Receptor Antagonists as New Therapeutic Tools for the Motivational Symptoms of Depression”, Front. Pharmacol., 9, 526 (2018), draw similar conclusions. Without being held to a particular explanation for its mechanism of action, it is believed that ETA indirectly activates ADORA2A receptors via neural circuits linked to the nasal olfactory chemosensory neurons in the olfactory epithelium.

Based in part on the discovery that ETA directly improves psychomotor and cognitive performance in mentally fatigued subjects with an efficacy similar to that of caffeine and modafinil, and its presumed method of action, ETA will similarly improve the psychomotor and cognitive performance of persons with mental fatigue associated with depressive disorders (such as MDD and peripartum depression).

In subjects experiencing decreased psychomotor and/or cognitive performance in mental fatigue associated with a depressive disorder such as MDD or peripartum depression, a therapeutically effective amount of ETA is nasally administered to the olfactory epithelium as described in Example 4. Psychomotor and cognitive performance improve in this group of subjects.

### Example 8—Improvement of Psychomotor and/or Cognitive Performance by ETA Supplementation in Subjects with Mental Fatigue Associated with Depressive Disorders Already Taking Antidepressant Drugs

Liu et al., “Low dose of caffeine enhances the efficacy of antidepressants in major depressive disorder and the underlying neural substrates”, Mol. Nutr. Food Res., 61, 8 (2017), have shown that supplemental administration of caffeine enhances the efficacy of common antidepressant drugs. For subjects taking escitalopram, Liu et al. reported that “chronic supplementation with a low dose of caffeine (60 mg) produced rapid antidepressant action by reduction of depressive scores. Furthermore, low doses of caffeine improved cognitive performance in depressed patients. However, caffeine did not affect sleep.” Evaluation metrics for attention, concentration, and working memory included a sustained attention task, i.e., target detection using tapping. Szopa et al., “Caffeine enhances the antidepressant-like activity of common antidepressant drugs in the forced swim test in mice”, Naunyn-Schmiedeber. Arch. Pharmacol., 389, 211-221 (2016), reported a similar enhancement by caffeine of the antidepressant-like activity of six typical antidepressants including imipramine, desipramine, fluoxetine, paroxetine, escitalopram, and reboxetine, in the forced swim test in mice, which is a widely used behavioral test to evaluate the antidepressant properties of drugs. Alexander et al., “Modafinil augmentation therapy in unipolar and bipolar depression: A systematic review and meta-analysis of randomized controlled trials”, J. Clin. Psych., 74(11), 1101-1107 (2013), have similarly shown that modafinil improves overall depression scores, remission rates and fatigue when used as adjunctive therapy for acute depressive episodes in both unipolar and bipolar disorders.

Based in part on the discovery that ETA directly improves psychomotor and cognitive performance in mentally fatigued subjects with an efficacy similar to that of caffeine and modafinil, and its presumed method of action, ETA will similarly improve the psychomotor and cognitive performance of persons with mental fatigue associated with depressive disorders (such as MDD and peripartum depression) when added to antidepressant treatment.

In subjects experiencing decreased psychomotor and/or cognitive performance in mental fatigue associated with MDD and already receiving treatment with escitalopram, a therapeutically effective amount of ETA is intranasally administered as described in Example 4 as a supplemental or adjunctive treatment. ETA is administered separately from the antidepressant treatment and is given sequentially or proximate in time to situations requiring attention and concentration. Depressive scores reduce beyond that of the escitalopram alone. Psychomotor and cognitive performance improve in this group of subjects.

### Example 9—Supplemental Administration of ETA with Itruvone Antidepressant Therapy

Itruvone (INN, pregn-4-en-20-yn-3-one) is being developed for the treatment of MDD and is an investigational product not approved by the Food and Drug Administration. The preparation and experimental treatment of MDD with this compound are described, for example, in Monti, U.S. patent Ser. No. 10/322,138, “Treatment of Depressive Disorders,”. Both itruvone and ETA are believed to have a common mechanism of action in terms of activating olfactory chemosensory neurons in the nasal mucosa, and then activating certain hypothalamic circuits in the brain without brain or systemic uptake. In subjects receiving itruvone treatment for major depressive disorder, ETA is administered to the olfactory epithelium as described in Example 4. Depressive scores reduce beyond that of itruvone alone. Cognitive function and psychomotor performance improve in this group of subjects. Because itruvone also is administered to the olfactory epithelium in the form of a spray or aerosol, ETA and itruvone may be administered separately, but proximate in time, or co-formulated for simultaneous intranasal administration.

### Example 10—Improvement of Psychomotor and/or Cognitive Performance by ETA in Subjects with Mental Fatigue Associated with Peripartum Depression

Peripartum depression (PPD, also referred to as postpartum depression; in the DSM-5 coding of depressive conditions as “with peripartum onset”, such as Major Depressive Disorder with peripartum onset) is one of the most prevalent peripartum complications and affects approximately 8-26% of peripartum women every year, and a negative association was reported between coffee intake and peripartum depression by Wang et al., “Coffee and caffeine intake and depression in postpartum women: A cross-sectional study from the National Health and Nutrition Examination Survey 2007-2018”, Front. Psychol., 14:1134522 (2023). Based in part on the discovery that ETA directly improves psychomotor and cognitive performance in mentally fatigued subjects with an efficacy similar to that of caffeine and modafinil, and its presumed method of action, ETA will similarly improve the psychomotor and cognitive performance of persons with mental fatigue associated with peripartum depression.

In subjects experiencing decreased psychomotor and/or cognitive performance associated with peripartum depression, a therapeutically effective amount of ETA is administered to the olfactory epithelium as described in Example 4. ETA is administered separately from any other antidepressant therapy and is given sequentially or proximate in time to such other medications. Depressive scores reduce when the subjects are evaluated. Psychomotor and cognitive performance improve in this group of subjects.

### Example 11—Improvement of Psychomotor and/or Cognitive Performance by ETA in Mental Fatigue Associated with Attention Deficit-Hyperactivity Disorder

Caffeine is known to be associated with improvements in cognition and attention in subjects with symptoms of ADHD. According to Cunha et al., “Potential therapeutic interest of adenosine A 2A receptors in psychiatric disorders,” Curr. Pharm. Des., 14(15), 1512-1524 (2008), the manipulation of ADORA2A receptors may be a novel and compelling novel therapeutic strategy to manage ADHD in view of the use of caffeine administration to treat this condition. Similarly, Heacock et al., U.S. Pat. No. 8,845,621, “Pharmaceutical Formulations of Modafinil”, report that modafinil at certain dosages significantly improved attention and significantly improved ADHD symptoms. Based in part on the discovery that ETA directly improves psychomotor and/or cognitive performance in mentally fatigued subjects with an efficacy similar to that of caffeine and modafinil, ETA will similarly improve the psychomotor and cognitive performance of persons with mental fatigue associated with ADHD.

In subjects experiencing decreased psychomotor and/or cognitive performance associated with ADHD, a therapeutically effective amount of ETA is administered intranasally as described in Example 4. Psychomotor and cognitive performance improve in this group of subjects.

### Example 12—Improvement of Psychomotor and/or Cognitive Performance by ETA in Mental Fatigue Associated with Mild Cognitive Impairment

Based in part on the discovery that ETA directly improves psychomotor and/or cognitive performance in mentally fatigued subjects with an efficacy similar to that of caffeine and modafinil, ETA will similarly improve the psychomotor and cognitive performance of persons with mental fatigue associated with MCI.

In subjects experiencing decreased psychomotor and/or cognitive performance associated with mild cognitive impairment, a therapeutically effective amount of ETA is administered intranasally as described in Example 4. Psychomotor and cognitive performance improve in this group of subjects.

1-29. (canceled)

30. A method of improving the psychomotor performance of a person with mental fatigue associated with sleep deprivation, comprising the intranasal administration of an effective amount of 1,3,5(10),16-estratetraen-3-yl acetate (ETA).

31. The method of claim 30, wherein the ETA is provided in a formulation suitable for administration as a nasal spray, a formulation suitable for administration as a nasal powder, or a formulation suitable for administration as a nasal aerosol.

32. The method of claim 31, wherein the nasal spray is administered to the nasal cavity from a nasal spray device comprising multiple doses of the ETA formulation and wherein the device releases about 400 ng to about 6000 ng of ETA per actuation of the nasal spray device.

33. The method of claim 30, wherein the sleep deprivation is associated with shift work sleep disorder or excessive daytime sleepiness.

34. A method of improving the cognitive performance of a person with mental fatigue associated with sleep deprivation, comprising the intranasal administration of an effective amount of 1,3,5(10),16-estratetraen-3-yl acetate (ETA).

35. The method of claim 34, wherein the ETA is provided in a formulation suitable for administration as a nasal spray, a formulation suitable for administration as a nasal powder, or a formulation suitable for administration as a nasal aerosol.

36. The method of claim 35, wherein the nasal spray is administered to the nasal cavity from a nasal spray device comprising multiple doses of the ETA formulation and wherein the device releases about 400 ng to about 6000 ng of ETA per actuation of the nasal spray device.

37. The method of claim 34, wherein the sleep deprivation is associated with shift work sleep disorder or excessive daytime sleepiness.

38. A method of improving the psychomotor performance of a person with mental fatigue associated with major depressive disorder (MDD), comprising the intranasal administration of an effective amount of 1,3,5(10),16-estratetraen-3-yl acetate (ETA).

39. The method of claim 38, wherein the ETA is provided in a formulation suitable for administration as a nasal spray, a formulation suitable for administration as a nasal powder, or a formulation suitable for administration as a nasal aerosol.

40. The method of claim 39, wherein the nasal spray is administered to the nasal cavity from a nasal spray device comprising multiple doses of the ETA formulation and wherein the device releases about 400 ng to about 6000 ng of ETA per actuation of the nasal spray device.

41. A method of improving the cognitive performance of a person with mental fatigue associated with major depressive disorder (MDD), comprising the intranasal administration of an effective amount of 1,3,5(10),16-estratetraen-3-yl acetate (ETA).

42. The method of claim 41, wherein the ETA is provided in a formulation suitable for administration as a nasal spray, a formulation suitable for administration as a nasal powder, or a formulation suitable for administration as a nasal aerosol.

43. The method of claim 42, wherein the nasal spray is administered to the nasal cavity from a nasal spray device comprising multiple doses of the ETA formulation and wherein the device releases about 400 ng to about 6000 ng of ETA per actuation of the nasal spray device.

44. A method of improving the psychomotor performance of a person with mental fatigue associated with attention deficit hyperactivity disorder (ADHD), comprising the intranasal administration of an effective amount of 1,3,5(10),16-estratetraen-3-yl acetate (ETA).

45. The method of claim 44, wherein the ETA is provided in a formulation suitable for administration as a nasal spray, a formulation suitable for administration as a nasal powder, or a formulation suitable for administration as a nasal aerosol.

46. The method of claim 45, wherein the nasal spray is administered to the nasal cavity from a nasal spray device comprising multiple doses of the ETA formulation and wherein the device releases about 400 ng to about 6000 ng of ETA per actuation of the nasal spray device.

47. A method of improving the cognitive performance of a person with mental fatigue associated with attention deficit hyperactivity disorder (ADHD), comprising the intranasal administration of an effective amount of 1,3,5(10),16-estratetraen-3-yl acetate (ETA).

48. The method of claim 47, wherein the ETA is provided in a formulation suitable for administration as a nasal spray, a formulation suitable for administration as a nasal powder, or a formulation suitable for administration as a nasal aerosol.

49. The method of claim 48, wherein the nasal spray is administered to the nasal cavity from a nasal spray device comprising multiple doses of the ETA formulation and wherein the device releases about 400 ng to about 6000 ng of ETA per actuation of the nasal spray device.

50. A method of improving the psychomotor performance of a person with mental fatigue associated with peripartum depression, comprising the intranasal administration of an effective amount of 1,3,5(10),16-estratetraen-3-yl acetate (ETA).

51. The method of claim 50, wherein the ETA is provided in a formulation suitable for administration as a nasal spray, a formulation suitable for administration as a nasal powder, or a formulation suitable for administration as a nasal aerosol.

52. The method of claim 51, wherein the nasal spray is administered to the nasal cavity from a nasal spray device comprising multiple doses of the ETA formulation and wherein the device releases about 400 ng to about 6000 ng of ETA per actuation of the nasal spray device.

53. A method of improving the cognitive performance of a person with mental fatigue associated with peripartum depression, comprising the intranasal administration of an effective amount of 1,3,5(10),16-estratetraen-3-yl acetate (ETA).

54. The method of claim 53, wherein the ETA is provided in a formulation suitable for administration as a nasal spray, a formulation suitable for administration as a nasal powder, or a formulation suitable for administration as a nasal aerosol.

55. The method of claim 54, wherein the nasal spray is administered to the nasal cavity from a nasal spray device comprising multiple doses of the ETA formulation and wherein the device releases about 400 ng to about 6000 ng of ETA per actuation of the nasal spray device.

56. A method of improving the psychomotor performance of a person with mental fatigue associated with mild cognitive impairment (MCI), comprising the intranasal administration of an effective amount of 1,3,5(10),16-estratetraen-3-yl acetate (ETA).

57. The method of claim 56, wherein the ETA is provided in a formulation suitable for administration as a nasal spray, a formulation suitable for administration as a nasal powder, or a formulation suitable for administration as a nasal aerosol.

58. The method of claim 57, wherein the nasal spray is administered to the nasal cavity from a nasal spray device comprising multiple doses of the ETA formulation and wherein the device releases about 400 ng to about 6000 ng of ETA per actuation of the nasal spray device.

59. A method of improving the cognitive performance of a person with mental fatigue associated with mild cognitive impairment (MCI), comprising the intranasal administration of an effective amount of 1,3,5(10),16-estratetraen-3-yl acetate (ETA).

60. The method of claim 59, wherein the ETA is provided in a formulation suitable for administration as a nasal spray, a formulation suitable for administration as a nasal powder, or a formulation suitable for administration as a nasal aerosol.

61. The method of claim 60, wherein the nasal spray is administered to the nasal cavity from a nasal spray device comprising multiple doses of the ETA formulation and wherein the device releases about 400 ng to about 6000 ng of ETA per actuation of the nasal spray device.

62. A method of treating a depressive disorder, comprising the administration of an effective amount of an antidepressant to a person who is also experiencing decreased psychomotor performance associated with mental fatigue, and further comprising, as an adjunctive treatment, intranasally administering an effective amount of 1,3,5(10),16-estratetraen-3-yl acetate (ETA) to improve the person's psychomotor performance.

63. The method of claim 62, wherein the antidepressant is itruvone nasal spray and is co-administered with the ETA.

64. A method of treating a depressive disorder, comprising the administration of an effective amount of an antidepressant to a person who is also experiencing decreased cognitive performance associated with mental fatigue, and further comprising, as an adjunctive treatment, intranasally administering an effective amount of 1,3,5(10),16-estratetraen-3-yl acetate (ETA) to improve the person's cognitive performance.

65. The method of claim 64, wherein the antidepressant is itruvone nasal spray and is co-administered with the ETA.
